(19) United States
(12) Patent Application Publication
(10) Pub. No.: US 2023/0190270 A1
(43) Pub. Date: Jun. 22, 2023

(54) Clip Device with Separable Connection and Having Variable Connection Force

(71) Applicant: MICRO-TECH (NANJING) CO., LTD., Nanjing, Jiangsu (CN)

(72) Inventors: Zhi Tang, Nanjing, Jiangsu (CN); Mingqiao Fan, Nanjing, Jiangsu (CN)

(21) Appl. No.: 17/921,817

(22) PCT Filed: Jun. 7, 2021
(86) PCT No.: PCT/CN2021/098588

§ 371 Date: Oct. 27, 2022

(30) Foreign Application Priority Data
Jun. 12, 2020 (CN) 202010534802.6

(51) Int. Cl. A61B 17/08; A61B 17/12

(52) U.S. Cl. CPC A61B 17/083; A61B 17/12013; A61B 2017/003

(57) ABSTRACT

A clip device with separable connection and having variable connection force, comprising a clipping portion and a delivery portion. The clipping portion comprises at least two side clipping arms and a clipping base. The side clipping arms are provided in the clipping base, and the proximal ends of the side clipping arms are connected to a control wire. A connection member for fixation and a connection member for separation are provided on the clipping portion. A distal end of the control wire is provided with a releaser, and the tail ends of the side clipping arms are connected to the releaser. During a clipping process, the side clipping arms may apply a larger gripping force on clipped tissues. During a releasing process, the control wire is ensured to apply a smaller dragging force, so as to separate the tail ends of the side clipping arms and the releaser.

The application claims priority to Chinese Patent Application No. 202010534802.6 filed on Jun. 12, 2020 by China Patent Office, entitled “CLIP WITH LARGE GRIPPING FORCE AND SMALL RELEASING FORCE”, which is incorporated herein by reference in its entirety.

### FIELD OF THE PRESENT DISCLOSURE

The present application relates to the technical field of endoscopic medical instruments, and in particular to a clip device with separable connection and having variable connection force.

### BACKGROUND OF THE PRESENT DISCLOSURE

A tissue clip device is a minimally invasive surgical instrument commonly used for hemostasis of wound surfaces in patients, such as hemostasis of digestive tract. The tissue clip device can be delivered to a surgical site in a patient’s body through an endoscopic working channel and can perform a clipping operation outside the body to complete hemostatic treatment.

Atypical tissue clip device includes a clipping portion, a delivery portion, and an operating component. In practice, the clipping portion and the delivery portion can be sent into the digestive tract of a patient through a endoscopic working channel, so that the clipping portion reaches a pre-determined surgical site in the patient’s body, and then surgical actions are performed through the operation component, and the performed surgical actions are then transmitted to the clipping portion through the delivery portion, so that the clipping portion generates an action response to perform hemostasis or clipping operation on the surgical wound surface.

The closure of the clipping portion and the separation of the clipping portion and the delivery portion are achieved by applying a proximal dragging action to a control wire via the operating component, and the whole dragging action can be divided into two force application processes. The first force is applied when the clipping portion is closed. In order to enable the clipping portion to clip the tissue, a greater connection force is required between the clipping portion and the delivery portion to ensure that the acting force applied by the surgical personnel can be effectively transmitted to the clipping portion. The second force is applied after the clipping portion is closed, whereby the clipping portion is separated from the delivery portion. In the conventional tissue clip device, when the first force is greater, the separable connection force is greater.

On the one hand, this type of separation uses a fixed and rated connection breaking force design, the separable connection undergoes a deformation when the applied force reaches a certain value, so that the clipping portion is separated from the delivery portion. For example, a structure of two thick end and a thin middle or a J-shaped hook is used. In practice, when the clipping portion performs the clipping action in a human body, a small axial travel cannot be sensed when the handle is operated by one or two hands to move towards the proximal end. When the clipping portion clips large tissue or hard tissue, even if the clipping action has not been completed, the clipping portion cannot continue to act due to resistance. However, at the moment, if the operator does not timely find the abnormality of the clipping portion, a large force is continuously applied to the clipping portion via the operating component outside the body, so that the separable connection portion is subjected to a large force and is deformed. In this way, when the clipping action is not completed, the clipping portion and the delivery portion may be separated in advance, resulting in the failure of the clipping action.

On the other hand, in order to enable the clipping portion to have a large clipping force, the clipping portion is designed to have a certain rigidity. In the process of actual operation, the force transmitted by the control wire during the process from opening to closing of the clipping portion needs to be large. However, when separating, the distance between the clipping portion and the operating component may exceed 2 m, and the natural bending of the human body may cause bending at the connection between the clipping portion and the delivery portion, so that the force state of the control wire has a positive pressure. The separable connection force of the distal end of the clipping portion transmitted to the operating component will be amplified several times, exceeding the stress limit of the operating personnel, and even the release force of the clipping portion will be excessive. As a result, the clipping portion cannot be effectively released through the operating component, and the clipping portion may be forcibly removed, causing bleeding injury to a patient.

### SUMMARY OF THE PRESENT DISCLOSURE

The present application provides a clip device with separable connection and having variable connection force, so as to solve the problem that when the clipping action of a conventional clip device is not completed, the clipping portion and the delivery portion are separated in advance, resulting in the failure of the clipping action, and to avoid excessive release force of the clipping portion, resulting in that the clipping portion cannot be effectively released by an operating component.

The present application provides a clip device with separable connection and having variable connection force, which comprises: a clipping portion and a delivery portion, wherein a proximal end of the clipping portion is detachably connected to a distal end of the delivery portion; the clipping portion comprises at least two side clipping arms and a clipping base; the delivery portion comprises a handle, a control wire and a sheath tube, the proximal end of the sheath tube is connected to the handle, and the control wire is provided inside the sheath tube;
the side clipping arms are provided in the clipping base, and the proximal end of the side clipping arm is connected to the control wire; the handle drives the control wire to move in the sheath tube, so that the side clipping arms move relative to the clipping base to open and close the side clipping arms; the clipping portion is provided with a connection member for fixation and a connection member for separation, a distal end of the control wire is provided with a releaser, and an end of the side clipping arm is connected to the releaser;
when the end of the side clipping arm is at the distal end of the clipping portion, the connection member for fixation constrains the separation action between the releaser and the end of the side clipping arm, so that the end of the side clipping arm and the releaser are in a fixed connection state; and
when the end of the side clipping arm is at a proximal end of the clipping portion, the connection member for separation is adapted to the releaser or the side clipping arm, so that the end of the side clipping arm and the releaser are in a separable connection state.

Optionally, when the end of the side clipping arm and the releaser are in a separable connection state, the connection force between the end of the side clipping arm and the releaser is a separable connection force;
when the end of the side clipping arm and the releaser are in a fixed connection state, the connection force between the end of the side clipping arm and the releaser is a fixed connection force;
the fixed connection force is greater than the separable connection force, and the separable connection force is greater than or equal to 5N.

Optionally, the connection between the releaser and the end of the side clipping arm is a deformable connection; when the end of the side clipping arm is at the distal end of the clipping portion, the connection member for fixation limits the deformation of the end of the side clipping arm or the releaser, so that the end of the side clipping arm and the releaser are in a fixed connection state;

when the end of the side clipping arm is at a proximal end of the clipping portion, the control wire drives the releaser or the distal end of the side clipping arm to disengage from the connection member for fixation, so as to separate the end of the side clipping arm from the releaser.

Optionally, the connection member for fixation is of a slot structure, the connection member for separation is a release cavity, the width of the slot is less than the width of the connection member for separation, and the connection member for fixation and the connection member for separation form a stepped groove.

Optionally, the releaser is provided thereon with a first connection hole, the first connection hole is connected to a first connection shaft at an end of a side clipping arm, and a first release port is provided at one side of a distal end of the first connection hole; the releaser is clamped into the connection member for fixation, and after the releaser moves from the connection member for fixation to the connection member for separation, the first release port is compressed and deformed and releases the first connection shaft.

Optionally, a guide bar is provided in the connection member for fixation, and a guide groove is provided on the releaser, and the guide groove clamps the guide bar.

Optionally, the end of the side clipping arm is provided with a second connection hole, the second connection hole is connected to a second connection shaft on the releaser, and a second release port is provided at one side of the proximal end of the second connection hole; a clamping block is further provided on the side clipping arm and is clamped in the connection member for fixation, and after the clamping block moves from the connection member for fixation to the connection member for separation, the second release port is compressed and deformed and releases the second connection shaft.

Optionally, the connection member for fixation is a snap fastener, and the snap fastener clamps the releaser so as to limit the deformation of the releaser, so that the end of the side clipping arm is in a fixed connection state with the releaser; the connection member for separation is a clamping block, wherein the clamping block is provided at the proximal end of the clipping portion and blocks the movement of the snap fastener towards the proximal end of the clipping portion, so that the snap fastener is disengaged from the releaser, and the end of the side clipping arm is in a separable connection state with the releaser.

Optionally, the connection mode between the releaser and the end of the side clipping arm is clamping; when the end of the side clipping arm is at the distal end of the clipping portion, the connection member for fixation is connected to the end of the side clipping arm, and the releaser is clamped, so that the end of the side clipping arm and the releaser are in a fixed connection state; and

when the end of the side clipping arm is at a proximal end of the clipping portion, the connection member for separation cooperates with the releaser, and the end of the side clipping arm and the releaser are in a separable connection state.

Optionally, the connection member for fixation is two circular arc-shaped clipping walls provided at the end of the side clipping arm, and a third release port formed between the two circular arc-shaped clipping walls is a connection member for separation; a third connection shaft is provided on the releaser, and the diameter of the third connection shaft is greater than an opening size of the third release port; when the end of the side clipping arm is at the distal end of the clipping portion, the connection member for fixation clamps the third connection shaft so that the end of the side clipping arm and the releaser are in a fixed connection state; and

when the end of the side clipping arm is at a proximal end of the clipping portion, the third connection shaft is at the third release port, and the distal end of the side clipping arm and the releaser are in a separable connection state.

Optionally, the connection member for fixation is a fourth connection shaft provided at the end of the side clipping arm, the releaser is provided with an arcuate slot, the fourth release port reserved by the arcuate slot is a connection member for separation, and the diameter of the fourth connection shaft is greater than an opening size of the fourth release port; when the end of the side clipping arm is at the distal end of the clipping portion, the fourth connection shaft clamps the arcuate slot, so that the end of the side clipping arm and the releaser are in a fixed connection state; and

when the end of the side clipping arm is at a proximal end of the clipping portion, the fourth connection shaft is at the fourth release port, and the end of the side clipping arm and the releaser are in a separable connection state.

Optionally, the connection member for fixation is a clamping bar, and an end of the side clipping arm and the releaser are provided with a clamping hole for clamping the clamping bar; the connection member for separation comprises an elastic member and an elastic block, and the elastic member is connected to the elastic block; when the end of the side clipping arm is at the distal end of the clipping portion, the clamping bar passes through and clamps the clamping hole of the end of the side clipping arm and the clamping hole of the releaser, so that the end of the side clipping arm and the releaser are in a fixed connection state; and

when the end of the side clipping arm is at a proximal end of the clipping portion, the elastic block pushes the clamping bar out of the clamping hole of the releaser, and the end of the side clipping arm is in a separable connection state with the releaser.

Optionally, the end of the side clipping arm is provided with a hook, and the distal end of the releaser is provided with a retaining wall and a fifth release port; wherein the connection member for fixation is a retaining wall, and the connection member for separation is a fifth release port;
the clipping base is provided with a protrusion, and the side surface of the releaser is further provided with a spiral groove cooperating with the protrusion; when the end of the side clipping arm is at the distal end of the clipping portion, the hook clamps the retaining wall, so that the end of the side clipping arm and the releaser are in a fixed connection state; and
when the end of the side clipping arm is at a proximal end of the clipping portion, the fifth release port rotates to the hook, and the end of the side clipping arm and the releaser are in a separable connection state.

Optionally, the clipping base is provided with a pin and the side clipping arm is provided with a motion slot, and the pin is slidable relative to the motion slot to control the opening and closing of the side clipping arm.

Optionally, the clipping portion further comprises a separation claw connected to the clipping base; the separation claw comprises a traction portion connected to the control wire, and a plurality of deformable connection claws provided on the traction portion;

the clipping base is provided with a plurality of holes for fixing the connection claw and for separating the connection claw from the clipping base when the tension of the control wire increases to the deformation limit of the connection claw. It can be seen from the above-mentioned technical solution that the present application provides a clip device with separable connection and having variable connection force. In practices, when the end of the side clipping arm is at the distal end of the clipping portion, the connection member for fixation constrains the separation of the releaser from the end of the side clipping arm, so that the end of the side clipping arm is in a fixed connection state with the releaser, ensuring that the side clipping arm has a greater gripping force. When the end of the side clipping arm is at a proximal end of the clipping portion, the connection member for separation is adapted to the releaser or the side clipping arm, so as to ensure that the control wire applies a small dragging force to achieve the separation of the end of the side clipping arm from the releaser.

It can be seen from the above-mentioned technical solution that the embodiment of the present application provides a clip device with separable connection and having variable connection force. In the process of clipping, when the end of the side clipping arm is at the distal end of the clipping portion, the connection member for fixation constrains the separation action between the releaser and the end of the side clipping arm, so that the end of the side clipping arm is in a fixed connection state with the releaser, and the side clipping arm can exert a greater gripping force on the clipped tissue, so as to ensure that the side clipping arm does not have a missed clipping during the process of clipping the tissue, so that the side clipping arm can certainly clip the tissue. In the releasing process, the end of the side clipping arm is at a proximal end of the clipping portion, and the connection member for separation is adapted to the releaser or the side clipping arm, so that the end of the side clipping arm is in a separable connection state with the releaser, so as to ensure that the control wire applies a small dragging force to achieve the separation of the end of the side clipping arm from the releaser, which is convenient for the operation personnel.

### BRIEF DESCRIPTION OF THE DRAWINGS

In order to explain the technical solution of the present application more clearly, the following will briefly introduce the drawings which need to be used in the embodiments, and it would be obvious for a person skilled in the art to obtain other drawings according to these drawings without involving any inventive effort.

FIG. 1 is a schematic diagram showing an overall structure of a clip device with separable connection and having variable connection force according to an embodiment of the present application;

FIG. 2 is a structure diagram of a pin cooperating with a motion slot according to an embodiment of the present application;

FIG. 3 is a structure diagram of an implementation A-1-1 according to an embodiment of the present application;

FIG. 4 is a structure diagram showing a state in which the side clipping arm of FIG. 3 is separated from the releaser;

FIG. 5 is a structure diagram of an implementation A-1-2 according to an embodiment of the present application;

FIG. 6 is a structure diagram of an implementation A-1-3 according to an embodiment of the present application;

FIG. 7 is a structure diagram of an implementation A-2 according to an embodiment of the present application;

FIG. 8 is a structure diagram of an implementation B-1 according to an embodiment of the present application;

FIG. 9 is another structure diagram of an implementation B-1 according to an embodiment of the present application;

FIG. 10 is a structure diagram of an implementation B-2 according to an embodiment of the present application;

FIG. 11 is a structure diagram of an implementation B-3 according to an embodiment of the present application;

FIG. 12 is a structure diagram of an implementation B-4 according to an embodiment of the present application;

FIG. 13 is a structure diagram showing a state in which a clipping portion and an input portion are completely separated according to an embodiment of the present application;

FIG. 14 is a structure diagram showing a coupling structure of a separation claw and a connection tube according to an embodiment of the present application.

List of reference numerals in the drawings:

| | |
|---|---|
| 1 | clipping portion |
| 11 | side clipping arm |
| 111 | first connection shaft |
| 112 | second connetion hole |
| 113 | second release port |
| 114 | clamping base |
| 115 | motion slot |
| 116 | hook |
| 12 | clipping base |
| 121 | pin |
| 122 | protrusion |
| 13 | connection member for fixation |
| 131 | guide bar |
| 14 | connection member for seperation |
| 15 | seperation claw |
| 151 | traction claw |
| 152 | connection claw |
| 2 | delivery portion |
| 21 | handle |
| 22 | control wire |
| 23 | sheath tube |
| 24 | releaser |
| 241 | first connection hole |
| 242 | first release port |
| 243 | guide groove |
| 244 | second connection shaft |
| 245 | third connection shaft |
| 246 | arcuate slot |
| 247 | spiral groove |

### DESCRIPTION OF THE EMBODIMENTS

It should be noted that in the technical solution provided in the present application, for the convenience of description, one end of the clip which is placed in the human body is referred to as a distal end, and the distal end is mainly used for performing a surgical action on the tissues of a human body. The end outside the body is referred to as a proximal end, and the proximal end is mainly used for operation by the operator. Unless otherwise stated in the present application, the distal end of each component is the end near the inside of the body, and the proximal end of each component is the end near outside of the body.

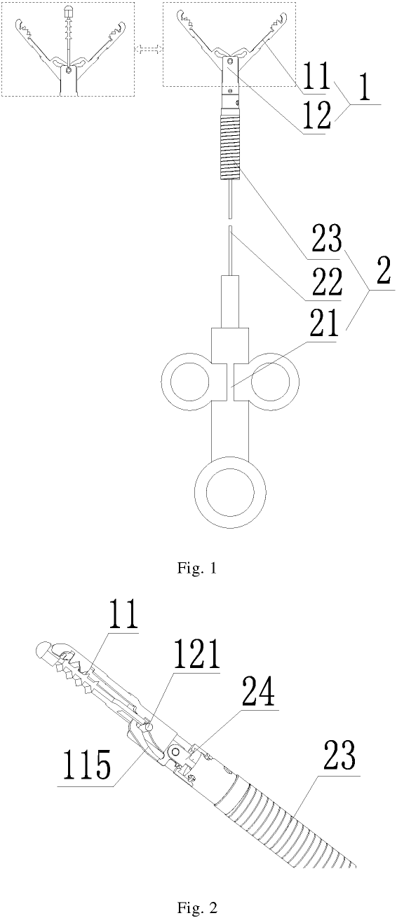

In order to prevent separating the clipping portion from the delivery portion in advance in the tissue clip device when the clipping action is not completed, resulting in the failure of the clipping action, and to avoid excessive release force of the clipping portion, resulting in that the clipping portion cannot be effectively released by an operating component, the embodiment of the present application provides a clip device with separable connection and having variable connection force as shown in FIG. 1, which comprises a clipping portion 1 and a delivery portion 2, wherein a proximal end of the clipping portion 1 is detachably connected to the distal end of the delivery portion 2.

The clipping portion 1 comprises at least two side clipping arms 11 and a clipping base 12. The delivery portion 2 comprises a handle 21, a control wire 22 and a sheath tube 23. The proximal end of the sheath tube is connected to the handle 21, and the control wire 22 is provided inside the sheath tube 23. The side clipping arm 11 is provided on the clipping base 12, and the proximal end of the side clipping arm 11 is connected to the control wire 22. The handle 21 drives the control wire 22 to move in the sheath tube 23, so as to move the side clipping arms 11 relative to the clip base 12, and to achieve the opening and closing of the side clipping arm 11.

The handle 21 is used for performing a surgical operation and is a direct control component of a surgical operator. In practice, the handle 21 drives the control wire 22 to move so as to control the opening and closing of the side clipping arms 11 of the clipping portion 1.

The proximal end of the sheath tube 23 is connected to the handle 21. In order to adapt to the structure of the human digestive tract and the shape of the endoscopic working channel, the sheath tube 23 adopts a soft circular tube structure, such as a plastic hose. In addition, a helically extending support spring is provided inside the sheath tube 23 to ensure that the sheath tube 23 can bear the compression of the lateral tissues of the digestive tract, so as to avoid the compression affecting the action of the devices inside the sheath tube 23. Embodiments of the present application provide a sheath tube 23 having a length that is sufficient for the handle 21 to perform an operation outside the body.

In order to achieve the movement of the side clipping arm 11 relative to the clipping base 12, as shown in FIG. 2, the clipping base 12 is provided with a pin 121, and the side clipping arm 11 is provided with a motion slot 115, wherein the pin 121 continuously passes through the motion slots 115 of the two side clipping arms 11 and can slide relative to the motion slot 115 so as to control the opening and closing of the side clipping arm 11.

It should be noted that, in practice, the pin 121 is fixedly provided on the clipping base 12 and the motion slot 115 moves relative to the clipping base 12. The motion slot 115 may have an L-shaped, obtuse-angled, arc-shaped or the like structure in order to achieve the closing of the clipping arm. In practice, taking a three-arm clip as an example, two side clipping arms 11 and a middle clip arm may be comprised, wherein the side clipping arms 11 generate a clipping action with respect to the middle clip arm under the action of the pin 121 and the motion slot 115 so as to clip the tissue. Further, as shown in FIG. 1, the side clipping arms 11 and the middle clipping arm are further provided with clipping teeth to enhance the tissue clipping ability of the side clipping arms 11 and the middle clipping arm.

It should be noted that the embodiment of the present application provides a clip with high gripping force and low releasing force, which may be a three-arm clip or a non-three-arm clip. As shown in FIG. 1, a view of a three-arm clip and a two-arm clip respectively is shown in a dotted box.

A connection member for fixation 13 and a connection member for separation 14 are further provided in the clipping portion 1, a releaser 24 is provided at the distal end of the control wire 22, and the distal end of the side clipping arm 11 is connected to the releaser 24.

In the process of actual application, when the control wire 22 drives the side clipping arm 11 to clip thick tissue and close, that is, when the end of the side clipping arm 11 is at the distal end of the clipping portion 1, the side clipping arm 11 is not completely closed, and the clipped thick tissue will also generate a greater resistance to the closure of the side clipping arm 11. At this time, the control wire 22 drives the side clipping arm 11 to continue to close. The separation action between the releaser 24 and the end of the side clipping arm 11 is limited by the restriction function of the connection member for fixation 13, so that the separation of the side clipping arm 11 from the control wire 22 in advance is avoided before the side clipping arm 11 is not completely closed. It needs to be noted that, the separation action of the side clipping arm 11 and the control wire 22 realizes the separation action between the side clipping arm 11 and the releaser 24 in physical structure, and the function realized is to separate the control relationship between the control wire 22 and the side clipping arm 11. Hereinafter, the separation between the control wire 22 and the side clipping arm 11 refers to separating the side clipping arm 11 and the releaser 24 in physical structure. Here, the connection member for fixation 13 can restrict the separating action of the releaser 24 and the end of the side clipping arm 11 by restricting the releaser 24 or the side clipping arm 11, wherein the restriction may be limiting the deformation of the releaser 24 or the side clipping arm 11, or may be limiting the position of the releaser 24 or the side clipping arm 11, whereby the releaser 24 and the end of the side clipping arm 11 cannot reach a separating state, namely, the end of the side clipping arm 11 is in a fixed connection state with the releaser 24. At the moment, the releaser 24 is inseparable from the end of the side clipping arm 11, ensuring that the side clipping arm 11 is able to withstand a greater dragging force of the control wire 22, so that the side clipping arm 11 may exert a greater gripping force on the clipped tissue without a case where the side clipping arm 11 becoming detached from the control wire 22.

During further closing of the side clipping arm 11, the handle 21 drives the control wire 22 to move in the sheath tube 23, so that the side clipping arm 11 moves relative to the clipping base 12. During the movement of the end of the side clipping arm 11 from the distal end to the proximal end, the opening of the side clipping arm 11 gradually becomes smaller, until the side clipping arm 11 is completely closed when the end of the side clipping arm 11 is at a proximal end of the clipping portion 1. At the moment, the connection member for separation 14 is adapted to the releaser 24 or the side clipping arm 11, so that the end of the side clipping arm 11 and the releaser 24 are in a separable connection state. At the moment, the operation end outside the body can determine that the side clipping arm 11 has been closed according to the extraction length of the control wire 22. The separation of the end of the side clipping arm 11 from the releaser 24 can be achieved with a relatively small force applied to the side clipping arm 11 via the control wire 22.

It should be noted that the adaptation referred to here may be that the position of the connection member for separation 14 corresponds to the position of the releaser 24 or the side clipping arm 11, or that the connection member for separation 14 moves in cooperation with the releaser 24 in such a way that the releaser 24 is in a position to be separated from the side clipping arm 11, or that the connection member for separation 14 disengages the releaser 24 or the side clipping arm 11 from the restraint of the connection member for fixation 13.

It can be seen from the above-mentioned technical solution that the embodiment of the present application provides a clip device with separable connection and having variable connection force. In the process of clipping, when the end of the side clipping arm 11 is at the distal end of the clipping portion 1, the connection member for fixation 13 constrains the separation action between the releaser 24 and the end of the side clipping arm 11, so that the end of the side clipping arm 11 is in a fixed connection state with the releaser 24, and the side clipping arm 11 can exert a greater gripping force on the clipped tissue, so as to ensure that the side clipping arm 11 does not have a missed clipping during the process of clipping the tissue, so that the side clipping arm 11 can certainly clip the tissue. In the releasing process, the end of the side clipping arm 11 is at a proximal end of the clipping portion 1, and the connection member for separation 14 is adapted to the releaser 24 or the side clipping arm 11, so that the end of the side clipping arm 11 is in a separable connection state with the releaser 24, so as to ensure that the control wire 22 applies a small dragging force to achieve the separation of the end of the side clipping arm 11 from the releaser 24, which is convenient for the operation personnel.

Furthermore, in an embodiment of the present application, when the end of the side clipping arm 11 and the releaser 24 are in a separable connection state, the connection force between the end of the side clipping arm 11 and the releaser 24 is a separable connection force. When the end of the side clipping arm 11 is in a fixed connection state with the releaser 24, the connection force between the end of the side clipping arm 11 and the releaser 24 is a fixed connection force. The separable connection force is greater than the fixed connection force, and the separable connection force is greater than or equal to 5N. It should be noted that, in practice, the magnitude of the separable connection force may be adapted to the application of the clip device. For example, the magnitude of the separable connection force may be adjusted by varying the rigidity of the releaser 24.

In an embodiment of the present application, in order to test the magnitude of the fixed connection force and the separable connection force, the following methods are used respectively.

Fixed connection force test method is described as follows. A handle 21 is moved so that an end of a side clipping arm 11 is at a distal end of a clipping portion 1, and at the moment, the side clipping arm 11 is in an open state. A connection member for fixation 13 constrains the separation action between a releaser 24 and the end of the side clipping arm 11 so that the end of the side clipping arm 11 and the releaser 24 are in a fixed connection state. A clipping base 12 is fixed to an upper clipping end of a tensile machine, and then a clipping object is placed in the clipping area of an expanded side clipping arm 11, so that the expanded side clipping arm 11 cannot be closed, ensuring that the end of the side clipping arm 11 is in a fixed connection state with the releaser 24. Then, the releaser 24 is fixed at a lower clipping end of the tensile machine, and the tensile machine is opened, wherein the tensile mode of tensile machine is used, and the speed of the tensile machine is set as 50 mm/min. The test procedure is started until the releaser 24 and the clipping base 12 are separated, and the maximum separation force between the releaser 24 and the clipping base 12 is recorded.

Detachable connection force test method is described as follows. A handle 21 is moved so that the end of the side clipping arm 11 is at a proximal end of the clipping portion 1, and at the moment, the side clipping arm 11 is in a closed state. A connection member for separation 14 is adapted to the releaser 24 or the side clipping arm 11 so that the end of the side clipping arm 11 is in a separable connection state with the releaser 24. The clipping base 12 is fixed to the upper clipping end of the tensile machine, with the side clipping arm 11 in a closed state. Then, the releaser 24 is fixed at a lower clipping end of the tensile machine, and the tensile machine is opened, wherein the tensile mode of tensile machine is used, and the speed of the tensile machine is set as 50 mm/min. The test procedure is started until the releaser 24 and the clipping base 12 are separated, and the maximum separation force between the releaser 24 and the clipping base 12 is recorded.

In order to achieve the connection and separation of the end of the side clipping arm 11 and the releaser 24, the connection mode of the releaser 24 and the end of the side clipping arm 11 is provided as a deformable connection in one implementation A of the present application. The deformation of the end of the side clipping arm 11 or the releaser is limited by means of the connection member for fixation 13, ensuring that the end of the side clipping arm 11 forms a fixation connection with the releaser 24. The control wire 22 drives the releaser 24 or the end of the side clipping arm 11 to disengage from the connection member for fixation 13, and the end of the side clipping arm 11 or the releaser 24 deforms to separate the end of the side clipping arm 11 from the releaser 24.

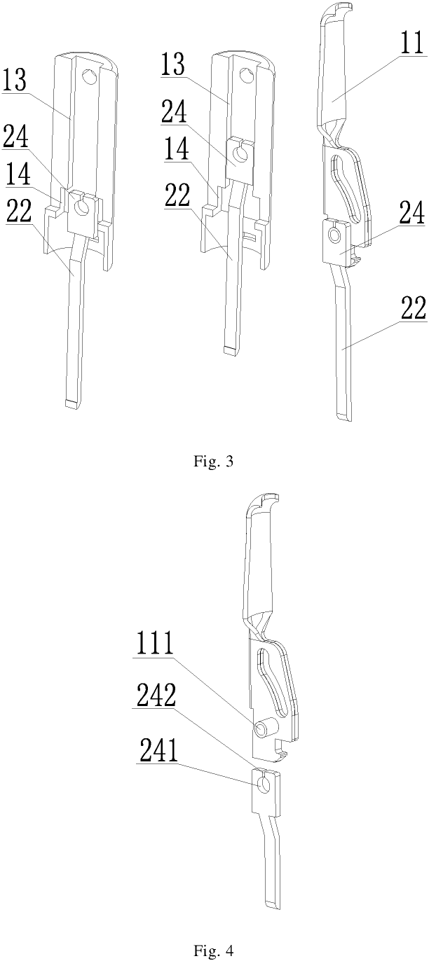

Specifically, in order to achieve that the connection member for fixation 13 restrains the releaser 24 from separating from the end of the side clipping arm 11, as shown in FIGS. 3, 4, 5 and 6, in the implementation A-1, the connection member for fixation 13 may be provided as a slot structure, the connection member for separation 14 may be provided as a release cavity, and the width of the slot is smaller than the width of the connection member for separation 14, so that the connection member for fixation 13 and the connection member for separation 14 constitute a stepped groove.

In the specific implementation, the deformable connection between the releaser 24 and the end of the side clipping arm 11 can be achieved in various ways. For example, in the implementation A-1-1, the releaser 24 is provided with a first connection hole 241, the first connection hole 241 is connected to the first connection shaft 111 at the end of the side clipping arm 11, and a first release port 242 is provided on one side of the distal end of the first connection hole 241.

The releaser 24 is clamped into the connection member for fixation 13, and after the releaser 24 moves from the inside of the connection member for fixation 13 to the inside of the connection member for separation 14, the first release port 242 is compressed and deformed and releases the first connection shaft 111.

As shown in FIG. 4, one side of the first connection hole 241 is not closed, and the first release port 242 is opened so that a side edge of the first connection hole 241 can be deformed. The side edge of the first connection hole 241 is clamped in the connection member for fixation 13, and the first connection hole 241 is connected to the first connection shaft 111. As shown in FIG. 3, when the end of the side clipping arm 11 is at the distal end of the clipping portion 1, the connection member for fixation 13 restricts the first connection shaft 111 from coming out of the first connection hole 241 so as to prevent the side clipping arm 11 from separating from the control wire 22 in advance. When the end of the side clipping arm 11 is at a proximal end of the clipping portion 1, the releaser 24 comes out of the restriction of the connection member for fixation 13 and enters the inside of the connection member for separation 14. At the moment, a side wall of the first connection hole 241 can be deformed, causing the first connection shaft 111 to slide out of the first release port 242 to achieve disengagement of the side clipping arm 11 from the control wire 22.

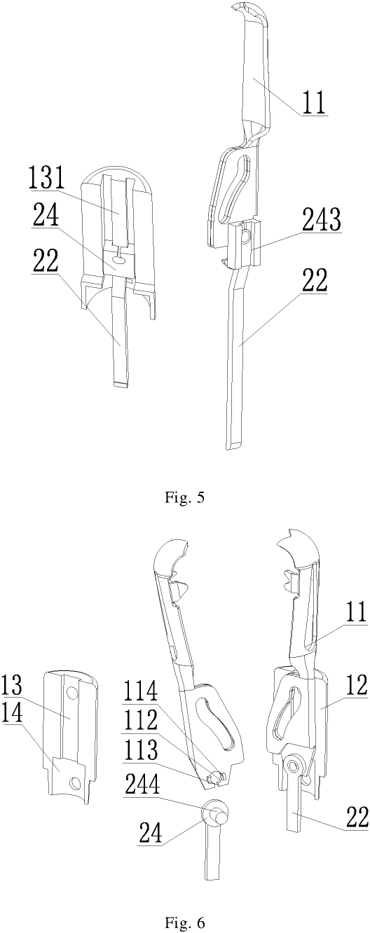

Furthermore, in order to ensure that the connection member for fixation 13 has a better guiding function so as to limit the movement of the releaser 24, as shown in FIG. 5, in the implementation A-1-2, a guide bar 131 is provided in the connection member for fixation 13, and a guide groove 243 is provided on the releaser 24. The guide groove 243 clamps the guide bar 131, and the sliding of the releaser 24 is ensured to be more stable by clamping between the guide groove 243 and the guide bar 131.

In order to achieve the deformable connection of the releaser 24 with the distal end of the side clipping arm 11, for example, as shown in FIG. 6, in the implementation A-1-3, the distal end of the side clipping arm 11 is provided with a second connection hole 112 connected with a second connection shaft 244 on the releaser 24. The proximal end side of the second connection hole 112 is provided with a second release port 113. A clamping block 114 is further provided on the side clipping arm 11, and the clamping block 114 is clamped in the connection member for fixation 13. After the clamping block 114 moves from the connection member for fixation 13 to the connection member for separation 14, the second release port 113 is compressed and deformed and releases the second connection shaft 244.

In the implementation A-1-3, when the end of the side clipping arm 11 is at the distal end of the clipping portion 1, the connection member for fixation 13 restricts the second connection shaft 244 from coming out of the second connection hole 112 by clamping the clamping block 114, so as to prevent the side clipping arm 11 from separating from the control wire 22 in advance. When the side clipping arm 11 reaches a separating position, the clamping block 114 comes out of the restriction of the connection member for fixation 13 and enters the connection member for separation 14. At the moment, the side wall of the second connection hole 112 can be deformed, causing the second connection shaft 244 to slid out of the second release port 113 to achieve disengagement of the side clipping arm 11 from the control wire 22.

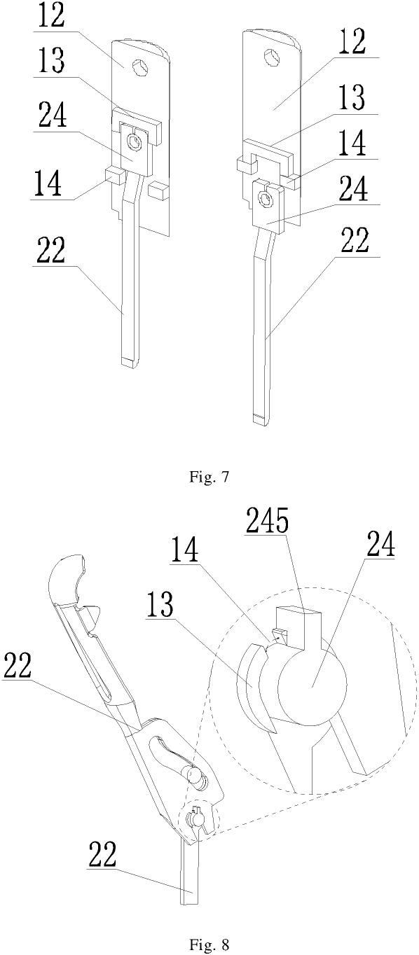

Specifically, in order to achieve that the connection member for fixation 13 restrains the releaser 24 from separating from the end of the side clipping arm 11, as shown in FIG. 7, in the implementation A-2, the connection member for fixation 13 is a snap fastener, and the connection member for separation 14 is a clamping block. The clamping block is provided at the proximal end of the clipping portion 1, and the position where the clamping block is provided is adapted to the separation position of the releaser 24. Namely, during the movement of the releaser 24 from the distal end to the proximal end of the clipping portion 1, the releaser 24 reaches the separation position of the releaser 24 from the side clipping arm 11 after passing through the clamping block.

When the end of the side clipping arm 11 is at the distal end of the clipping portion 1, the releaser 24 is clamped by the snap fastener and follows the movement of the releaser 24, and the deformation of the releaser 24 is restricted, so that the end of the side clipping arm 11 forms a fixed connection with the releaser 24.

When the end of the side clipping arm 11 is at a proximal end of the clipping portion 1, the clamping block forms a blocking action on the snap fastener, so that the snap fastener is disengaged from the releaser 24. After the control wire 22 continues to apply a dragging force, the releaser 24 is pulled and deformed, thereby achieving the separation of the end of the side clipping arm 11 from the releaser 24.

In order to achieve the connection and separation of the end of the side clipping arm 11 and the releaser 24, in another implementation B of the present application, the connection mode of the releaser 24 and the end of the side clipping arm 11 is clamping. When the end of the side clipping arm 11 is at the distal end of the clipping portion 1, the connection member for fixation 13 is connected to the end of the side clipping arm 11, and the releaser 24 is clamped so that the end of the side clipping arm 11 and the releaser 24 are in a fixed connection state. When the end of the side clipping arm 11 is at a proximal end of the clipping portion 1, the connection member for separation 14 cooperates with the releaser 24, and the distal end of the side clipping arm 11 and the releaser 24 are in a separable connection state.

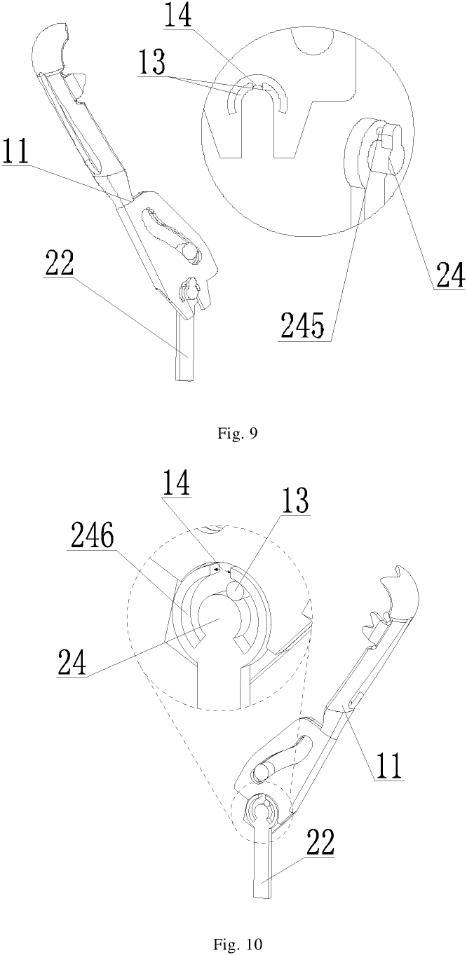

In a specific implementation process, clamping between the releaser 24 and the end of the side clipping arm 11 can be achieved in various ways. For example, as shown in FIGS. 8 and 9, in implementation B-1, the connection member for fixation 13 is two segments of circular arc-shaped clipping walls provided at the end of the side clipping arm 11, and a third release port formed between the two segments of circular arc-shaped clipping walls is a connection member for separation 14. A third connection shaft 245 is provided on the releaser 24, and the diameter of the third connection shaft 245 is greater than an opening size of the third release port.

When the end of the side clipping arm 11 is at the distal end of the clipping portion 1, the third connection shaft 245 moves in a circular arc-shaped clipping wall area on one side, and forms a fixed connection between the end of the side clipping arm 11 and the releaser 24 through a clamping fit.

When the end of the side clipping arm 11 is at a proximal end of the clipping portion 1, the third connection shaft 245 moves to a third release port between two segments of the two circular arc-shaped clipping walls. After the control wire 22 continues to apply a dragging force, the third connection shaft 245 is extruded out from the third release port so as to achieve the separation of the end of the side clipping arm 11 from the releaser 24. It needs to be explained that in order to ensure that the third connection shaft 245 can be smoothly extruded out from the fourth release port without causing the third connection shaft 245 to naturally fall off from the third release port, the diameter of the third connection shaft 245 is set to be slightly larger than the opening size of the third release port.

Specifically, in the implementation B-1, two modified structures are provided in FIGS. 8 and 9. In FIG. 8, the circular arc-shaped clipping wall is a side surface provided at the end of the side clipping arm 11. In FIG. 9, the circular arc-shaped clipping wall is a hollow structure provided at the end of the side clipping arm 11.

For another example, as shown in FIG. 10, in the implementation B-2, the connection member for fixation 13 is a fourth connection shaft provided at the end of the side clipping arm 11. The releaser 24 is provided with an arcuate slot 246, a fourth release port reserved by the arcuate slot 246 is a connection member for separation 14, and the diameter of the fourth connection shaft is greater than an opening size of the fourth release port. When the end of the side clipping arm 11 is at the distal end of the clipping portion 1, the fourth connection shaft moves along the arcuate slot 246, so that the end of the side clipping arm 11 forms a fixed connection with the releaser 24 through a clamping action.

When the end of the side clipping arm 11 is at a proximal end of the clipping portion 1, the fourth connection shaft moves to the fourth release port, and the end of the side clipping arm 11 is in a separable connection state with the releaser 24. When the control wire 22 continues to apply a dragging force, the fourth connection shaft is extruded out from the fourth release port so as to achieve the separation of the end of the side clipping arm 11 from the releaser 24. It should be noted that, to ensure that the fourth connection shaft can be smoothly extruded out from the fourth release port without causing the fourth connection shaft to naturally fall off from the fourth release port, the diameter of the fourth connection shaft is set to be slightly larger than the opening size of the fourth release port.

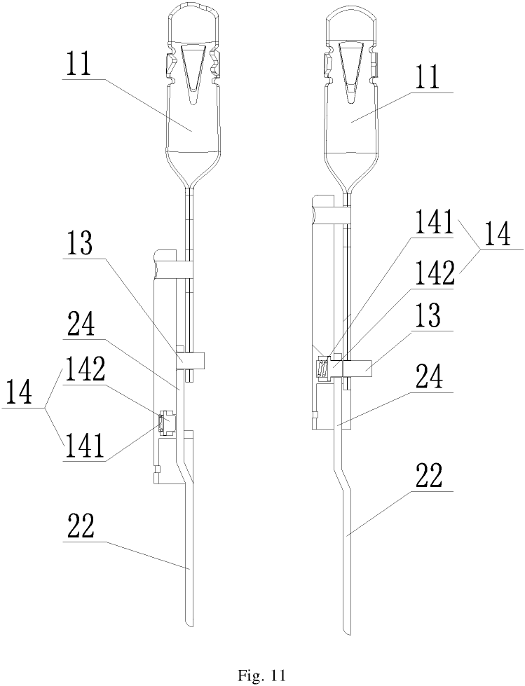

For another example, as shown in FIG. 11, in implementation B-3, the connection member for fixation 13 is a clamping bar, and a clamping hole for clamping the clamping bar is provided on the end of the side clipping arm 11 and the releaser 24. The connection member for separation 14 includes an elastic member 141 and an elastic block 142, and the elastic member 141 is coupled to the elastic block 142. Specifically, the elastic member 141 may be a spring.

When the end of the side clipping arm 11 is at the distal end of the clipping portion 1, the clamping bar passes through the clamping hole at the end of the side clipping arm 11 and the clamping hole on the releaser 24 in sequence, and forms a fixed connection between the end of the side clipping arm 11 and the releaser 24 through a clamping action.

When the end of the side clipping arm 11 is at a proximal end of the clipping portion 1, one end of the clamping bar moves to the position of the elastic block 142. The elastic block 142 is pressed by the elastic member 141, and the clamping bar is pushed out of the clamping hole of the releaser 24. On the side of the elastic block 142 close to the side clipping arm 11, an inclined plane is provided. After the control wire 22 continues to apply a dragging force, the edge of the clamping hole on the releaser 24 presses the inclined plane, so that the elastic block is disengaged from the clamping hole of the releaser 24, achieving the separation of the end of the side clipping arm 11 from the releaser 24.

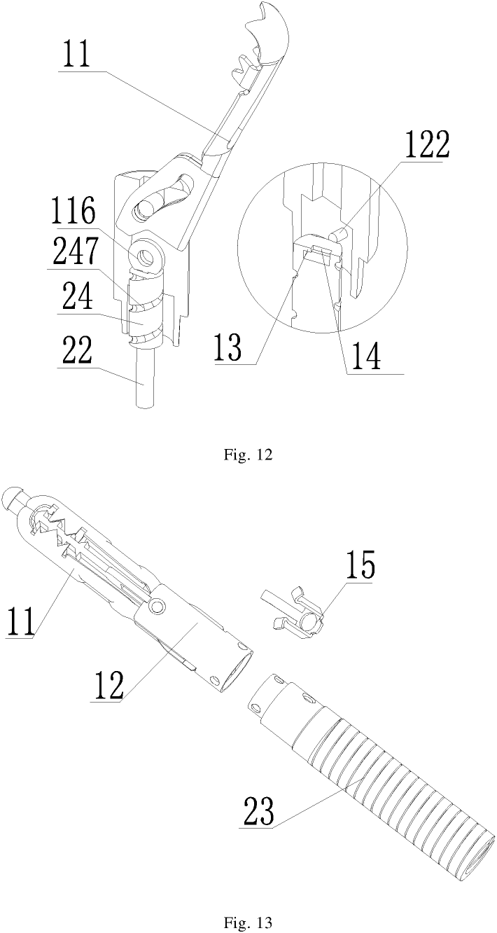

For another example, as shown in FIG. 12, in the implementation B-4, the end of the side clipping arm 11 is provided with a hook 116, and the distal end of the releaser 24 is provided with a retaining wall and a fifth release port, wherein the connection member for fixation 13 is the retaining wall, and the connection member for separation 14 is the fifth release port. The clipping base 12 is provided with a protrusion 122, and the side surface of the releaser 24 is further provided with a spiral groove 247 cooperating with the protrusion 122. When the end of the side clipping arm 11 is at the distal end of the clipping portion 1, the hook 116 clamps the retaining wall so that the end of the side clipping arm 11 is in a fixed connection state with the releaser 24.

When the end of the side clipping arm 11 is at a proximal end of the clipping portion 1, the fifth release port rotates to the hook 116, and the end of the side clipping arm 11 is in a separable connection state with the releaser 24. When the end of the side clipping arm 11 is at the distal end of the clipping portion 1, the hook 116 clamps the retaining wall, and the end of the side clipping arm 11 is in a fixed connection with the releaser 24.

Note that as used herein, the hook 116 being engaged with the retaining wall means that the hook body at the tail of the hook 116 is clamped with the retaining wall, and the hook 116 being adapted to the fifth release port means that the hook body at the tail of the hook 116 can be released from the fifth release port.

When the end of the side clipping arm 11 is at a proximal end of the clipping portion 1, the spiral groove 247 contacts and clamps the protrusion 122. When pressed by the protrusion 122, the releaser 24 rotates. After the fifth release port rotates to the position of the hook 116, the control wire 22 continues to apply a dragging force, and the hook 116 is released from the fifth release port, thereby achieving the separation of the end of the side clipping arm 11 from the releaser 24.

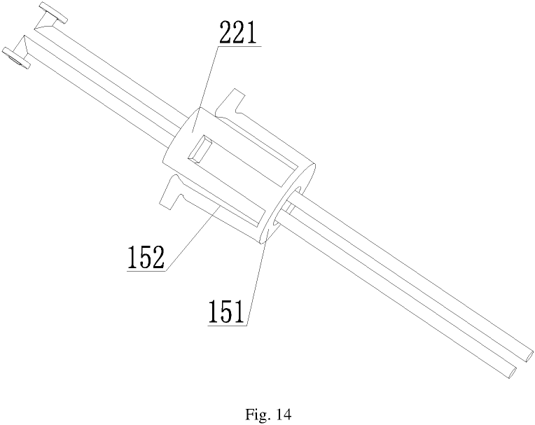

In order to achieve a separable connection between the clipping portion 4 and the sheath tube 23, as shown in FIGS. 13 and 14, in some embodiments of the present application, the clipping portion 1 further comprises a separation claw 15 connecting the clipping base 12. The separation claw 15 comprises a traction portion 151 connected to the control wire 22, and a plurality of deformable connection claws 152 provided on the traction portion 151. The clipping base 12 is provided with a plurality of holes for fixing the connection claw 152 and for separating the connection claw 152 from the clipping base 12 when the tension of the control wire 22 increases to the deformation limit of the connection claw 152.

A connection tube 221 is provided on the control wire 22, and the control wire 22 penetrates the connection tube 221. The connection tube 221 contacts the traction portion 151 to pull the separation claw 15. In the present embodiment, the separation claw 15 has a plurality of connection claws 152 in the circumferential direction. The connection claws 152 can be engaged with the hole portions of the clipping base 12 and the sheath tube 23. Any part of the traction portion 151 is simultaneously connected to the control wire 22, and the connection claws 152 deform under the tension of the control wire 22 to disengage the clipping base 12 and the sheath tube 23, so as to achieve an effective separation of the clipping base 12 and the sheath tube 23.

It can be seen from the above-mentioned technical solution that the embodiment of the present application provides a clip device with separable connection and having variable connection force. In the process of clipping, when the end of the side clipping arm 11 is at the distal end of the clipping portion 1, the connection member for fixation 13 constrains the separation action between the releaser 24 and the end of the side clipping arm 11, so that the end of the side clipping arm 11 is in a fixed connection state with the releaser 24, and the side clipping arm 11 can exert a greater gripping force on the clipped tissue, so as to ensure that the side clipping arm 11 does not have a missed clip during the process of clipping the tissue, so that the side clipping arm 11 can certainly clip the tissue. In the releasing process, the end of the side clipping arm 11 is at a proximal end of the clipping portion 1, and the connection member for separation 14 is adapted to the releaser 24 or the side clipping arm 11, so that the end of the side clipping arm 11 is in a separable connection state with the releaser 24, so as to ensure that the control wire 22 applies a small dragging force to achieve the separation of the end of the side clipping arm 11 from the releaser 24, which is convenient for the operation personnel.

Similar elements between the embodiments provided herein may be referred to each other. The specific embodiments provided above are merely exemplary of the general concepts of the present application and are not intended to limit the scope of the present application. For a person skilled in the art, any other embodiment obtained according to the present application without inventive effort falls within the scope of the present application.

1. A clip device with separable connection and having variable connection force, comprising: a clipping portion and a delivery portion, a proximal end of the clipping portion being detachably connected to a distal end of the delivery portion;
wherein the clipping portion comprises at least two side clipping arms and a clipping base; the delivery portion comprises a handle, a control wire and a sheath tube; a proximal end of the sheath tube is connected to the handle, and the control wire is provided inside the sheath tube;
wherein the side clipping arms are provided in the clipping base, and a proximal end of the side clipping arm is connected to the control wire; the handle drives the control wire to move in the sheath tube so that the side clipping arms move relative to the clipping base to open and close the side clipping arms;
wherein the clipping portion is provided with a connection member for fixation and a connection member for separation, a distal end of the control wire is provided with a releaser, and an end of the side clipping arm is connected to the releaser;
when the end of the side clipping arm is at the distal end of the clipping portion, the connection member for fixation constrains a separation action between the releaser and the end of the side clipping arm, so that the end of the side clipping arm and the releaser are in a fixed connection state; and
when the end of the side clipping arm is at the proximal end of the clipping portion, the connection member for separation is adapted to the releaser or the side clipping arm, so that the end of the side clipping arm and the releaser are in a separable connection state.

2. The clip device with separable connection and having variable connection force according to claim 1, wherein the connection force between the end of the side clipping arm and the releaser is a separable connection force when the end of the side clipping arm and the releaser are in a separable connection state;
wherein the connection force between the end of the side clipping arm and the releaser is a fixed connection force when the end of the side clipping arm and the releaser are in a fixed connection state; and
wherein the fixed connection force is greater than the separable connection force, and the separable connection force is greater than or equal to 5N.

3. The clip device with separable connection and having variable connection force according to claim 1, wherein the connection between the releaser and the end of the side clipping arm is a deformable connection;
when the end of the side clipping arm is at the distal end of the clipping portion, the connection member for fixation limits the deformation of the end of the side clipping arm or the releaser, so that the end of the side clipping arm and the releaser are in a fixed connection state;
when the end of the side clipping arm is at a proximal end of the clipping portion, the control wire drives the releaser or the end of the side clipping arm to disengage from the connection member for fixation, so as to separate the end of the side clipping arm from the releaser.

4. The clip device with separable connection and having variable connection force according to claim 3, wherein the connection member for fixation is of a slot structure, the connection member for separation is a release cavity, a width of the slot is less than that of the connection member for separation, and the connection member for fixation and the connection member for separation form a stepped groove.

5. The clip device with separable connection and having variable connection force according to claim 3, wherein a first connection hole is provided on the releaser, the first connection hole is connected to a first connection shaft at the end of the side clipping arm, and a first release port is provided at one side of a distal end of the first connection hole;
wherein the releaser is clamped in the connection member for fixation, and after the releaser moves from the connection member for fixation to the connection member for separation, the first release port is compressed and deformed and releases the first connection shaft.

6. The clip device with separable connection and having variable connection force according to claim 3, wherein a guide bar is provided in the connection member for fixation and a guide groove is provided on the releaser, and the guide groove clamps the guide bar.

7. The clip device with separable connection and having variable connection force according to claim 3, wherein a second connection hole is provided at the end of the side clipping arm and is connected to a second connection shaft on the releaser, and a second release port is provided at one side of the proximal end of the second connection hole;
wherein a clamping block is further provided on the side clipping arm and is clamped in the connection member for fixation, and after the clamping block moves from the connection member for fixation to the connection member for separation, the second release port is compressed and deformed and releases the second connection shaft.

8. The clip device with separable connection and having variable connection force according to claim 3, wherein the connection member for fixation is a snap fastener, and the snap fastener clamps the releaser to limit the deformation of the releaser, so that the end of the side clipping arm is in a fixed connection state with the releaser;
wherein the connection member for separation is a clamping block, the clamping block is provided at the proximal end of the clipping portion and blocks the movement of the snap fastener towards the proximal end of the clipping portion, so that the snap fastener is disengaged from the releaser, and the end of the side clipping arm is in a separable connection state with the releaser.

9. The clip device with separable connection and having variable connection force according to claim 1, wherein the connection mode between the releaser and the end of the side clipping arm is clamping;
when the end of the side clipping arm is at the distal end of the clipping portion, the connection member for fixation is connected to the end of the side clipping arm, and the releaser is clamped, so that the end of the side clipping arm and the releaser are in a fixed connection state; and
when the end of the side clipping arm is at a proximal end of the clipping portion, the connection member for separation cooperates with the releaser, and the end of the side clipping arm and the releaser are in a separable connection state.

10. The clip device with separable connection and having variable connection force according to claim 1, wherein the connection member for fixation is two circular arc-shaped clipping walls provided at the end of the side clipping arm, and a third release port formed between the two circular arc-shaped clipping walls is a connection member for separation; a third connection shaft is provided on the releaser, and the diameter of the third connection shaft is greater than an opening size of the third release port;
when the end of the side clipping arm is at the distal end of the clipping portion, the connection member for fixation clamps the third connection shaft so that the end of the side clipping arm and the releaser are in a fixed connection state; and
when the end of the side clipping arm is at a proximal end of the clipping portion, the third connection shaft is at the third release port, and the end of the side clipping arm and the releaser are in a separable connection state.

11. The clip device with separable connection and having variable connection force according to claim 1, wherein the connection member for fixation is a fourth connection shaft provided at the end of the side clipping arm, the releaser is provided with an arcuate slot, a fourth release port reserved by the arcuate slot is a connection member for separation, and the diameter of the fourth connection shaft is greater than an opening size of the fourth release port;
when the end of the side clipping arm is at the distal end of the clipping portion, the fourth connection shaft clamps the arcuate slot, so that the end of the side clipping arm and the releaser are in a fixed connection state; and
when the end of the side clipping arm is at a proximal end of the clipping portion, the fourth connection shaft is at the fourth release port, and the end of the side clipping arm and the releaser are in a separable connection state.

12. The clip device with separable connection and having variable connection force according to claim 1, wherein the connection member for fixation is a clamping bar, and a clamping hole for clamping the clamping bar is provided on the end of the side clipping arm and the releaser; the connection member for separation comprises an elastic member and an elastic block, and the elastic member is connected with the elastic block;
when the end of the side clipping arm is at the distal end of the clipping portion, the clamping bar passes through and clamps the clamping hole of the end of the side clipping arm and the clamping hole on the releaser, so that the end of the side clipping arm and the releaser are in a fixed connection state; and
when the end of the side clipping arm is at a proximal end of the clipping portion, the elastic block pushes the clamping bar out of the clamping hole of the releaser, and the end of the side clipping arm and the releaser are in a separable connection state.

13. The clip device with separable connection and having variable connection force according to claim 1, wherein a hook is provided at the end of the side clipping arm, and the distal end of the releaser is provided with a retaining wall and a fifth release port; wherein the connection member for fixation is a retaining wall, and the connection member for separation is a fifth release port;
wherein the clipping base is provided with a protrusion, and the side surface of the releaser is further provided with a spiral groove cooperating with the protrusion ;
when the end of the side clipping arm is at the distal end of the clipping portion, the hook clamps the retaining wall, so that the end of the side clipping arm and the releaser are in a fixed connection state; and
when the end of the side clipping arm is at a proximal end of the clipping portion, the fifth release port rotates to the hook, and the end of the side clipping arm and the releaser are in a separable connection state.

14. The clip device with separable connection and having variable connection force according to claim 1, wherein the clipping base is provided with a pin and the side clipping arm is provided with a motion slot, and the pin is slidable with respect to the motion slot to control the opening and closing of the side clipping arm.

15. The clip device with separable connection and having variable connection force according to claim 1, wherein the clipping portion further comprises a separation claw connecting to the clipping base; wherein the separation claw comprises a traction portion connected to the control wire, and a plurality of deformable connection claws provided on the traction portion;
wherein the clipping base is provided with a plurality of holes for fixing the connection claw and for separating the connection claw from the clipping base when the tension of the control wire increases to the deformation limit of the connection claw.

16. The clip device with separable connection and having variable connection force according to claim 9, wherein the connection member for fixation is two circular arc-shaped clipping walls provided at the end of the side clipping arm, and a third release port formed between the two circular arc-shaped clipping walls is a connection member for separation; a third connection shaft is provided on the releaser, and the diameter of the third connection shaft is greater than an opening size of the third release port;
when the end of the side clipping arm is at the distal end of the clipping portion, the connection member for fixation clamps the third connection shaft so that the end of the side clipping arm and the releaser are in a fixed connection state; and
when the end of the side clipping arm is at a proximal end of the clipping portion, the third connection shaft is at the third release port, and the end of the side clipping arm and the releaser are in a separable connection state.

17. The clip device with separable connection and having variable connection force according to claim 9, wherein the connection member for fixation is a fourth connection shaft provided at the end of the side clipping arm, the releaser is provided with an arcuate slot, a fourth release port reserved by the arcuate slot is a connection member for separation, and the diameter of the fourth connection shaft is greater than an opening size of the fourth release port;
when the end of the side clipping arm is at the distal end of the clipping portion, the fourth connection shaft clamps the arcuate slot, so that the end of the side clipping arm and the releaser are in a fixed connection state; and
when the end of the side clipping arm is at a proximal end of the clipping portion, the fourth connection shaft is at the fourth release port, and the end of the side clipping arm and the releaser are in a separable connection state.

18. The clip device with separable connection and having variable connection force according to claim 9, wherein the connection member for fixation is a clamping bar, and a clamping hole for clamping the clamping bar is provided on the end of the side clipping arm and the releaser; the connection member for separation comprises an elastic member and an elastic block, and the elastic member is connected with the elastic block;
when the end of the side clipping arm is at the distal end of the clipping portion, the clamping bar passes through and clamps the clamping hole of the end of the side clipping arm and the clamping hole on the releaser, so that the end of the side clipping arm and the releaser are in a fixed connection state; and
when the end of the side clipping arm is at a proximal end of the clipping portion, the elastic block pushes the clamping bar out of the clamping hole of the releaser, and the end of the side clipping arm and the releaser are in a separable connection state.

19. The clip device with separable connection and having variable connection force according to claim 9, wherein a hook is provided at the end of the side clipping arm, and the distal end of the releaser is provided with a retaining wall and a fifth release port; wherein the connection member for fixation is a retaining wall, and the connection member for separation is a fifth release port;
wherein the clipping base is provided with a protrusion, and the side surface of the releaser is further provided with a spiral groove cooperating with the protrusion;
when the end of the side clipping arm is at the distal end of the clipping portion, the hook clamps the retaining wall, so that the end of the side clipping arm and the releaser are in a fixed connection state; and
when the end of the side clipping arm is at a proximal end of the clipping portion, the fifth release port rotates to the hook, and the end of the side clipping arm and the releaser are in a separable connection state.
